(19) United States
(12) Patent Application Publication
(10) Pub. No.: US 2025/0083128 A1
(43) Pub. Date: Mar. 13, 2025

(54) SINTERING-RESISTANT METAL CATALYST SUPPORTED ON SINGLE ATOMIC CE-DOPED METAL OXIDES, METHOD OF PREPARING SAME, AND USE THEREOF OF PREPARING SAME, AND USE THEREOF

(71) Applicants: KOREA ADVANCED INSTITUTE OF SCIENCE AND TECHNOLOGY, Daejeon (KR); HANWHA SOLUTIONS CORPORATION, Seoul (KR)

(72) Inventors: Min Kee CHOI, Daejeon (KR); Han Chang KWON, Daejeon (KR)

(21) Appl. No.: 18/727,350

(22) PCT Filed: Jan. 11, 2023
(86) PCT No.: PCT/KR2023/000467

§ 371 Date: Jul. 9, 2024

(30) Foreign Application Priority Data
Jan. 11, 2022 (KR) 10-2022-0004132

(51) Int. Cl. B01J 23/10; B01J 21/04; B01J 35/30; B01J 37/04; B01J 37/08; B01J 38/04; C07C 2/24; C07C 5/02

(52) U.S. Cl. CPC B01J 23/10; B01J 21/04; B01J 35/30; B01J 37/04; B01J 37/08; B01J 38/04; C07C 2/24; C07C 5/02; B01J 2235/30

(57) ABSTRACT

The present invention relates to a sintering-resistant metal catalyst supported on a single-atomic Ce-doped metal oxide and use thereof, and more particularly to a sintering-resistant metal catalyst, a method for preparing same, and use thereof, wherein the metal catalyst has improved resistance to sintering while maintaining metallicity of the metal even in high temperature thermochemical reaction by including an active metal component in a metal oxide carrier doped with single atomic sized Ce. The metal catalyst has improved resistance to sintering while maintaining metallicity of the active metal, and thus exhibits excellent catalytic performance in a high-temperature thermochemical reaction, such as aromatization, dehydrogenation or hydrogenation of hydrocarbons.

### TECHNICAL FIELD

The present invention relates to a sintering-resistant metal catalyst supported on a single-atomic Ce-doped metal oxide and use thereof, and more particularly to a sintering-resistant metal catalyst, a method for preparing same, and use thereof, wherein the metal catalyst has improved resistance to sintering while maintaining metallicity of the metal even in high temperature thermochemical reaction by including an active metal component in a metal oxide carrier doped with single atomic sized Ce.

### RELATED ART

Heterogeneous metal catalysts are widely used in many processes in the refining and petrochemical industries and play a pivotal role in the modern chemical and energy industries.

Due to the nature of the mass device industry, where a single plant can produce hundreds to millions of tons of chemicals per year, the lifetime is of paramount importance in a newly developed catalyst when it is applied to real-world processes. The opportunity cost of replacing a catalyst once is very high in real industry, so catalysts currently in industrial use have lifetimes of a few months to a few years. Most metal catalysts exhibit deactivation by sintering, a phenomenon in which metal particles agglomerate during high-temperature chemical processes. This reduces the activity of the catalyst as fewer metals are available on the exposed surface to participate in the reaction, resulting in a short catalyst lifetime.

In response thereto, academia and industry have made various efforts to suppress the sintering of metal catalysts (Want et al., Adv. Mater. 2019, 31, 1901905; Goodman et al., ACS Catal. 2017, 7, 7156). Strategies being explored include the development of carriers with enhanced binding to metals (Wang et al., Nat. Commun. 2020, 11, 529; Hatanaka et al., J. Catal. 2009, 266, 182; Liu et al., Nat. Commun. 2019, 10, 5790; Qiao et al., Nat. Chem. 2011, 3, 634), immobilization of metal particles using a coating layer (Kim et al., ChemCatChem 2019, 11, 4653; Lu et al., ACS Catal. 2020, 10, 13957), and immobilization of metal particles using a microporous carrier (Zhang et al., Appl. Surf. Sci. 2019, 494, 1044; PCT/KR2020/003914).

Among the above methods, immobilization strategies using a coating layer and a microporous carrier have the advantage of physically inhibiting the mobility of the metal atoms or particles, thus effectively preventing the sintering of the metal. However, the accessibility of reactants to the metal surface is also reduced, which entails a sacrifice of catalytic activity.

On the other hand, using a carrier that can form a strong bond with the metal has the advantage of minimizing the reduction in accessibility of the metal particles, along with the relative ease of catalyst preparation. Carriers that can form strong bonds with metals are known to include metal oxides such as alumina (Al₂O₃), ceria (CeO₂), titania (TiO₂), and iron oxide (FeOₓ). Precious metals such as platinum (Pt), palladium (Pd), and rhodium (Rh) can be resistant to sintering because they are located at defect sites in metal oxide carriers and form strong bonds with neighboring oxygen atoms. However, the strong bonding of these metals to the carrier involves the transfer of electrons, which increases the oxidation number of the metal. Since most metal catalysts used in the refining or petrochemical industry have the reduced form of the metal as their active point, oxidized forms of metal catalysts exhibit low catalytic activity.

To overcome the above problems, a catalyst in the form where a metal oxide carrier is supported with not only a precious metal but also a second metal or a semi-metal having a large electronegativity has been reported (Korean Patent Publication No. 10-2008-0025142). It is reported that, in the above catalyst, the bond between the precious metal and oxygen is weakened by the second metal or semi-metal, which can suppress the tendency of the precious metal to be oxidized.

Accordingly, the inventors of the present invention have made a good faith effort to solve the above problems and to prepare a catalyst with excellent resistance to sintering while maintaining metallicity of the metal. As a result, they have found that, if a catalyst, where active metal components in an alumina (Al₂O₃) carrier doped with single atomic sized Ce, is synthesized, a metal catalyst having excellent activity, selectivity, and stability with improved resistance to sintering while maintaining metallicity of the metal even in high temperature thermochemical reaction can be prepared, which can be used in high temperature thermochemical reaction such as aromatization, hydrogenation, and dehydrogenation, and have completed the present invention.

### PRIOR ART DOCUMENT

### [Patent Document]

Korean Patent Publication No. 10-2008-0025142

### Non-Patent Document

Miho Hatanaka et al., Reversible changes in the Pt oxidation state and nanostructure on a ceria-based supported Pt, Journal of Catalysis 266 (2009) 182-190 Seunghyun Kim et al., Sintering Resistance of Pt@SiO2 Core-Shell Catalyst, ChemCatChem 11 (2019) 4653-4659
Sheng Lu et al., Atomic Layer Deposition Overcoating Improves Catalyst Selectivity and Longevity in Propane Dehydrogenation, ACS Catalysis 10 (2020) 13957-13967
Zeshu Zhang et al., Anti-sintering Pd@silicalite-1 for methane combustion: Effects of the moisture and SO₂, Applied Surface Science 494 (2019) 1044-1054

### SUMMARY OF THE INVENTION

It is an object of the present invention to provide a metal catalyst having excellent activity, selectivity and stability, in which the sintering occurring at high temperature is effectively suppressed while metallicity of the active metal is maintained, and a method for preparing the same.

Another object of the present invention is to provide use for the metal catalysts in high temperature thermochemical reaction such as aromatization, hydrogenation and dehydrogenation.

To accomplish the above objectives, the present invention provides a metal catalyst comprising a metal oxide carrier doped with single atomic Ce; and an active metal component supported on the metal oxide carrier.

The present invention also provides a method of preparing the metal catalyst, comprising (a) mixing a Ce precursor, a metal oxide precursor, and an active metal component precursor; (b) aging the mixture obtained in step (a); and (c) drying and heat treating the mixture obtained in step (b).

The present invention also provides a method of aromatization using a metal catalyst, comprising reacting a saturated hydrocarbon in the presence of the metal catalyst to produce an aromatic compound.

The present invention also provides a dehydrogenation method using a metal catalyst, comprising reacting a saturated hydrocarbon in the presence of the metal catalyst to produce an olefin compound.

The present invention also provides a method of hydrogenation using a metal catalyst, comprising reacting a hydrocarbon in the presence of the metal catalyst to produce a hydrogenated compound.

### BRIEF DESCRIPTION OF DRAWING

FIG. 1 is a diagram illustrating a sintering-resistant metal catalyst prepared by supporting a metal on a single-atomic Ce-doped alumina (Al₂O₃) carrier, in accordance with examples of the present invention.

FIG. 2 is a high-angle annular dark-field scanning transmission electron microscopy (HAADF-STEM) image of a single-atomic Ce-doped alumina carrier (Ce—Al₂O₃), according to Example 1 of the present invention.

FIG. 3 shows the results of Ce_{3d} X-ray photoelectron spectroscopy (XPS) measurements of an alumina carrier doped with single-atomic Ce (Ce—Al₂O₃) and an alumina carrier containing ceria (CeO₂—Al₂O₃), according to Example 1 of the present invention.

FIG. 4 is a graph showing the aromatic compound yields during the n-octane aromatization reaction and catalyst regeneration cycle, according to Example 2 of the present invention.

FIG. 5 is a HAADF-STEM image of catalysts subjected to n-octane aromatization reaction and catalyst regeneration, according to Example 3 of the present invention.

FIG. 6 shows the results of Pt Lmn-edge X-ray absorption near edge structure (XANES) analysis of catalysts subjected to n-octane aromatization reaction and catalyst regeneration, according to Example 3 of the present invention.

FIG. 7 is a graph showing the propylene yield during a propane dehydrogenation and catalyst regeneration cycle, according to Example 4 of the present invention.

FIG. 8 is a graph showing the turnover rate of the benzene hydrogenation per mol of platinum, according to Example 5 of the present invention.

FIG. 9 is a HAADF-STEM image of Pd/Ce—Al₂O₃ and Pd/CeO₂—Al₂O₃ that have undergone the calcination process, according to Example 6 of the present invention.

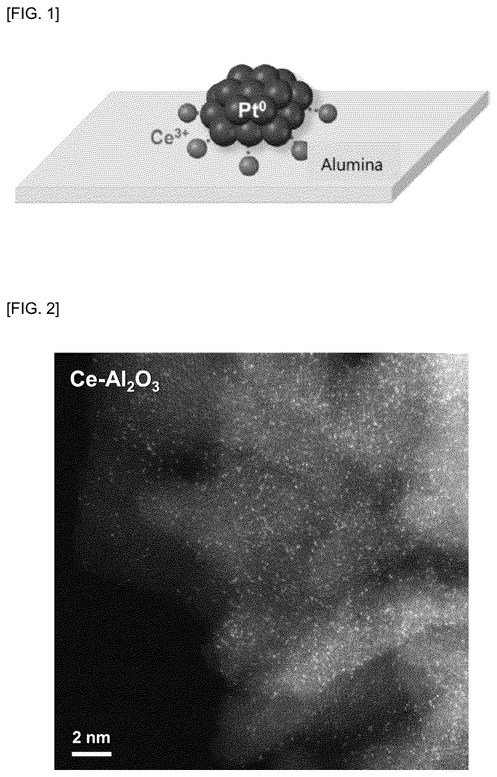

### DETAILED DESCRIPTION OF THE INVENTION AND PREFERRED EXAMPLES

Unless otherwise defined, all technical and scientific terms used herein have the same meaning as would be commonly understood by a person skilled in the art. In general, the nomenclature used herein is well-known and in common use in the art.

The present invention has confirmed that when a catalyst containing active metal components is synthesized on an alumina (Al₂O₃) carrier doped with single atomic sized Ce to prepare a catalyst with excellent resistance to sintering while maintaining metallicity of the metal, a metal catalyst having excellent activity, selectivity, and stability with enhanced resistance to sintering can be prepared and used in high temperature thermochemical reaction such as aromatization, hydrogenation, and dehydrogenation.

Accordingly, the present invention relates, in one aspect, to a metal catalyst comprising a metal oxide carrier doped with single-atomic Ce; and an active metal component supported on the metal oxide carrier.

The present invention relates, in another aspect, to a method of preparing the metal catalyst comprising (a) mixing a Ce precursor, a metal oxide precursor, and an active metal component precursor; (b) aging the mixture obtained in step (a) above; and (c) drying and heat treating the mixture obtained in step (b) above.

The present invention is described in detail below.

A metal catalyst according to the present invention includes a metal oxide carrier doped with single-atomic Ce; and an active metal component supported on the metal oxide carrier.

As used in the present invention, the term “carrier” refers to a solid material that disperses and stabilizes a catalytically functional material and is highly dispersed to increase the exposed surface area of the catalytically functional material. It is usually porous, large in area, and mechanically, thermally, and chemically stable.

Also, as used herein, the term “support” refers to the adherence of a catalytic component to the surface of a carrier. In general, the terms immobilization, adsorption, binding, and dispersion can be used interchangeably.

According to one preferred embodiment of the present invention, there is provided an alumina (Al₂O₃) carrier doped with a single atom of Ce; and a metal catalyst comprising an active metal component in the carrier.

In the present invention, the catalyst exhibits the following characteristics in the Ce_{3d}-XPS spectrum:

(Area of the u′″ peak)/(Total area of the Ce_{3d} spectrum)×100(%)<1(%)

In the above, u′″ refers to the characteristic peak of Ce⁴⁺ that appears at 917 eV in the Ce_{3d}-XPS spectrum.

In the present invention, the Ce content of the carrier may be 0.01% by weight to 5% by weight relative to the metal catalyst.

In the present invention, the metal oxide may be at least one selected from the group consisting of alumina (Al₂O₃) and aluminate (MAl₂O₄; wherein M is at least one selected from Mg, Cr, Mn, Fe, Co, Ni, and Zn), preferably alumina.

In the present invention, the active metal component may be one or more of platinum (Pt), palladium (Pd), ruthenium (Ru), rhodium (Rh), iridium (Ir), iron (Fe), cobalt (Co), nickel (Ni), molybdenum (Mo), manganese (Mn), gold (Au), silver (Ag), copper (Cu), gallium (Ga), and zinc (Zn).

In the present invention, the content of the active metal component may be 0.01% by weight to 10% by weight.

According to preferred examples of the metal catalyst of the present invention, Ce—Al₂O₃, Ce—Al₂O₃, PtGa/Ce—Al₂O₃, Pd/Ce—Al₂O₃ can be used.

According to preferred examples of the present invention, there is provided a method for the preparation of a metal catalyst comprising an active metal component in an alumina carrier doped with monatomic Ce, comprising (a) mixing cerium (Ce), aluminum (AI) and an active metal precursor; (b) aging the mixture; and (c) drying and heat treating (calcining) the mixture.

In the present invention, the Ce precursor in step (a) above may be one or more selected from the group consisting of cerium nitrate, cerium fluoride, cerium phosphate, cerium chloride, and cerium sulfate.

In the present invention, the aluminum (AI) precursor of the metal oxide in step (a) above may be one or more selected from the group consisting of aluminum alkoxide, aluminum nitrate, aluminum fluoride, aluminum phosphate, aluminum chloride, aluminum sulfate, boehmite, and pseudo-boehmite. Furthermore, the metal precursor other than aluminum of the metal oxide may be one or more selected from the group consisting of nitrate, chloride, or sulfate compounds of magnesium (Mg), chromium (Cr), manganese (Mn), iron (Fe), cobalt (Co), nickel (Ni), or zinc (Zn).

In the present invention, in step (c) above, the drying may be performed at a temperature of 50 to 200° C., and the heat treatment may be performed at a temperature of 350 to 1100° C.

In another aspect, the present invention relates to a method for aromatizing or dehydrogenating or hydrogenating hydrocarbons in the presence of a metal catalyst containing an active metal component on the single-atomic Ce doped alumina carrier.

The aromatization reaction of n-octane was carried out in the presence of the metal catalyst of the present invention to produce benzene, toluene, xylene, and ethylbenzene in superior yields compared to conventional catalysts, and it was found that the catalyst exhibited high catalytic stability.

Accordingly, the present invention relates to a method of aromatization using a metal catalyst, comprising performing an aromatization reaction of a saturated hydrocarbon in the presence of the metal catalyst to produce an aromatic compound, in accordance with one example.

In the present invention, the saturated hydrocarbon may be pentane, hexane, heptane or octane, and the aromatic compound produced may be benzene, toluene, xylene or ethylbenzene. The post-reaction products include, in addition to the main products, by-products obtained by hydrogenolysis and cracking, which can be separated by further purification methods such as gas chromatography.

In addition, the aromatization reaction of octane and catalyst regeneration cycling experiments were carried out in the presence of the metal catalyst of the present invention, and it was found that the single-atomic Ce inhibited the sintering of Pt through strong bonding with Pt, thereby exhibiting excellent catalytic performance.

Accordingly, the present invention relates, in another aspect, to a method for simultaneously performing aromatization and catalyst regeneration using a metal catalyst, comprising the following steps:
(a) carrying out a primary aromatization reaction of a saturated hydrocarbon in the presence of a metal catalyst according to the present invention and flowing helium;
(b) regenerating the metal catalyst with dry air and flowing with helium;
(c) in-situ reduction in a hydrogen atmosphere; and
(d) performing a secondary aromatization reaction.

Furthermore, the dehydrogenation of propane was carried out in the presence of the metal catalyst of the present invention to produce propylene in a superior yield compared to conventional catalysts, and it was confirmed that the catalyst exhibited high catalytic stability at the same time.

Accordingly, the present invention relates to a method of dehydrogenation using a metal catalyst, comprising performing a dehydrogenation of a saturated hydrocarbon in the presence of the metal catalyst to produce an olefin compound, according to another aspect of the present invention.

In the present invention, the saturated hydrocarbon may be ethane, propane, n-butane, i-butane, butene, or cyclohexane.

In another aspect, the present invention relates to a method for simultaneously performing dehydrogenation and catalyst regeneration using a metal catalyst, comprising:
(a) performing a primary dehydrogenation of saturated hydrocarbons in the presence of a metal catalyst according to the present invention and flowing helium;
(b) regenerating the metal catalyst with dry air and flowing with helium; and
(c) performing a secondary dehydrogenation.

It was also confirmed that the hydrogenation of benzene under a hydrogen atmosphere in the presence of the metal catalyst of the present invention can be carried out to produce cyclohexane, and the product is cyclohexane, the main product, without any other by-products, thus exhibiting a high benzene conversion rate compared to conventional catalysts and thus exhibiting excellent catalytic activity.

Accordingly, in accordance with another aspect, the present invention relates to a method of hydrogenation using a metal catalyst, comprising performing a hydrogenation of an aromatic hydrocarbon or an unsaturated hydrocarbon under a hydrogen atmosphere in the presence of the metal catalyst to produce an alicyclic or aliphatic compound.

The hydrogenation of benzene under a hydrogen atmosphere in the presence of a metal catalyst can be carried out to produce the product cyclohexane. However, no by-products other than the main product, cyclohexane, were observed in the product, and it exhibits high benzene conversion rate compared to conventional catalysts, thereby exhibiting excellent catalytic activity.

The following examples are used herein as examples of hydrogenation: 1) hydrogenation, 2) hydrodesulfurization, 3) hydrodenitrogenation, 4) hydrodeoxygenation, and 5) hydroisomerization. Each reaction uses different reactants, so there are a wide variety of reactants available. Representative reactants and products for each reaction are shown below.

### 1. Hydrogenation

A wide variety of unsaturated hydrocarbons are available as reactants in hydrogenation. Examples of preferred hydrogenation reactions include the following reactions:
Acetylene->Ethylene
Propyne->Propylene
Butadiene->Butene
Vinylacetylene->Butadiene

### 2. Hydrodesulfurization

Saturated and unsaturated hydrocarbons, including sulfur, are possible as reactants in hydrodesulfurization. Examples of preferred hydrodesulfurization include the following reactions:
Thiophene->Butane+Hydrogen sulfide
Benzothiophene->Ethylene benzene+Hydrogen sulfide
Dibenzothiophene->Biphenyl+Hydrogen sulfide

### 3. Hydrodenitrogenation

Saturated and unsaturated hydrocarbons, including nitrogen, are possible as reactants in hydrodenitrogenation. Examples of preferred hydrodenitrogenation include the following reactions:
Pyrrole->Butane+Ammonia
Pyridine->Pentane+Ammonia
Quinoline->Propyl cyclohexane+Ammonia

### 4. Hydrodeoxygenation

Saturated and unsaturated hydrocarbons, including oxygen, are possible as reactants in hydrodeoxygenation. Examples of preferred hydrodeoxygenation include the following reactions:
Furan->Butane+Water
Dibenzofuran->Biphenyl+Water

### 5. Hydroisomerization

Hydrogenation isomerization can produce isomers using normal saturated hydrocarbons in the diesel (10-15 carbon number) range and paraffins in the lubricating oil (20-40 carbon number) range as reactants.

In the present invention, the hydrogenation may be hydrogenation, hydrodesulfurization, hydrodenitrogenation, hydrodeoxygenation, or hydroisomerization.

The reaction conditions for the aromatization, dehydrogenation and hydrogenation, e.g. temperature, pressure range, time range or presence of gas, can be varied without any restrictions. Preferably, the dehydrogenation can be carried out at a temperature of 550-650° C. and a pressure range of 0.1-1 atm.

Hereinafter, preferred examples of the present invention are described for the purpose of illustrating the present invention, but it will be apparent to those skilled in the art that various changes and modifications are possible within the scope of the present invention and the technical idea, and that such changes and modifications fall within the scope of the appended patent claims.

### EXAMPLES

### Manufacturing Example 1: Synthesis of Single-Atomic Ce-Doped Alumina Carrier (Ce—Al₂O₃)

To 200 mL of distilled water, 0.70 g of cerium nitrate (Ce(NO₃)₃) was added and stirred at room temperature until completely dissolved. 2.3 g of an aqueous solution of nitric acid (HNO₃) (61%) and 23 g of aluminum isopropoxide (C₉H₂₁O₃Al) were then sequentially added to the stirred aqueous solution. The content of Ce in the above mixture was fixed at 5% by weight based on the final carrier. The mixed solution was refluxed at 100° C. for 12 hours and then dried at 80° C. for 12 hours. The dried solid particles were recovered and calcined in an oxygen atmosphere at 750° C. for 2 hours. The prepared carrier was referred as “Ce—Al₂O₃.”

### Comparative Example 1: Synthesis of Alumina Carrier with Ceria (CeO₂—Al₂O₃)

To 200 mL of distilled water, 3.4 g of cerium nitrate (Ce(NO₃)₃) was added and stirred at room temperature until completely dissolved. 2.3 g of an aqueous solution of nitric acid (HNO₃) (61%) and 23 g of aluminum isopropoxide (C₉H₂₁O₃Al) were then sequentially added to the stirred aqueous solution. The content of Ce in the above mixture was fixed at 20% by weight based on the final carrier. The mixed solution was refluxed at 100° C. for 12 hours and then dried at 80° C. for 12 hours. The dried solid particles were recovered and calcined in an oxygen atmosphere at 750° C. for 2 hours. The prepared carrier was referred as “CeO₂—Al₂O₃.”

### Example 1: Analysis of Structures and Characteristics of Alumina Carriers Containing Ce

To determine the dispersion state of Ce in the Ce—Al₂O₃ carrier synthesized by Manufacturing Example 1 above, high-angle annular dark-field scanning transmission electron microscopy (HAADF-STEM) analysis was performed, and the results are shown in FIG. 2.

As shown in FIG. 2, the Ce—Al₂O₃ of Manufacturing Example 1 shows that the single atomic-sized Ce atoms are evenly dispersed in the alumina (Al₂O₃) carrier.

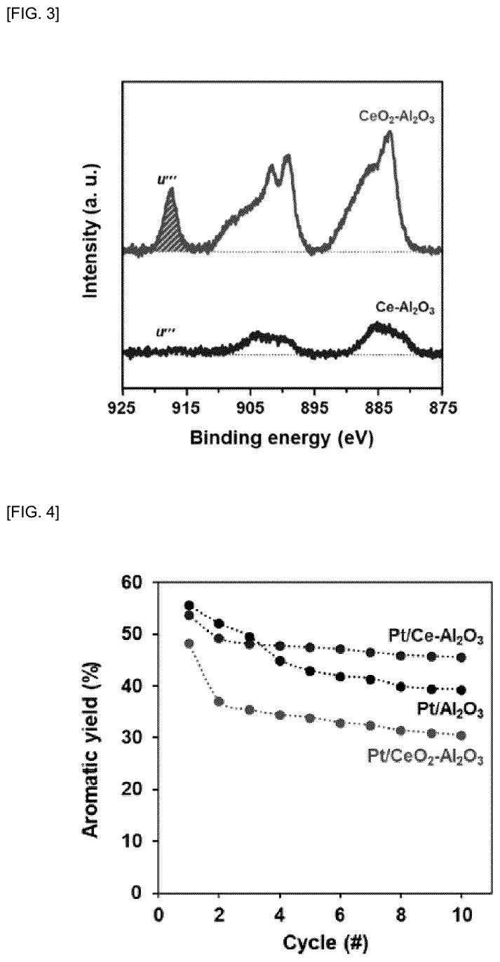

In addition, Ce_{3d} X-ray photoelectron spectroscopy (XPS) analysis was performed to determine the oxidation state of Ce present in the Ce—Al₂O₃ and CeO₂—Al₂O₃ carriers synthesized in Manufacturing Example 1 and Comparative Example 1, and the results are shown in FIG. 3 and Table 1.

As shown in FIG. 3, the u′″ peak (917 eV), a characteristic peak exhibited by Ce⁴⁺, was hardly observed in Ce—Al₂O₃ of Manufacturing Example 1, while a clear u′″ peak was observed in CeO₂—Al₂O₃ of Comparative Example 1. For quantitative analysis of the u′″ peaks, the area ratios of the u′″ peaks of the above carriers were calculated from Mathematical Formula 1 below and are shown in Table 1.

$$Area ratio of the ````peak ( \% ) = \frac{Area of the u```` peak}{Total area of the {Ce}_{3 d} spectrum} \times 100 \qquad [ Mathematical Formula 1 ]$$

TABLE 1

| | Area ratio of the ″″ peak (%) |
|---|---|
| Manufacturing Example 1 (Ce—Al₂O₃) | 0.3 |
| Comparative Example 1 (CeO₂—Al₂O₃) | 9.2 |

As shown in Table 1 above, the Ce—Al₂O₃ of Manufacturing Example 1 exhibited a very low area ratio of the u′″ peak (0.3%), which means that the single atomic Ce present in the carrier is mostly in the form of Ce³⁺.

### Manufacturing Example 2: Synthesis of Pt/Ce—Al₂O₃ Using a Single-Atomic Ce Doped Alumina Carrier

To 200 mL of distilled water, 0.70 g of cerium nitrate (Ce(NO₃)₃), and 0.12 g of tetraamineplatinum nitrate ((NH₃)₄Pt(NO₃)₂), were added and stirred at room temperature until completely dissolved. Then 2.3 g of aqueous nitric acid (HNO₃) solution (61%) and 23 g of aluminum isopropoxide (C₉H₂₁O₃Al) were added sequentially to the stirred aqueous solution. The contents of Ce and Pt in the above mixture were fixed at 5% by weight and 1% by weight, respectively, based on the final catalyst. The mixed solution was refluxed at 100° C. for 12 hours and then dried at 80° C. for 12 hours. The dried solid particles were recovered and calcined in an oxygen atmosphere at 750° C. for 2 hours. The prepared catalyst was referred as “Pt/Ce—Al₂O₃.”

### Comparative Example 2: Synthesis of Pt/Al₂O₃ Using Commercial Alumina as a Carrier

0.2 g of tetraamineplatinum nitrate, (NH₃)₄Pt(NO₃)₂ was dissolved in 6.0 g of distilled water and then supported on 10 g of γ-Al₂O₃(Strem Chemicals) by wet-impregnation method. The content of Pt was fixed at 1% by weight based on the final catalyst. After drying at 80° C. for 12 hours, it was calcined in an oxygen atmosphere at 750° C. for 2 hours. The prepared catalyst was referred as “Pt/Al₂O₃.”

### Comparative Example 3: Synthesis of Pt/CeO₂—Al₂O₃ Using Alumina Carrier Containing Ceria

To 200 mL of distilled water, 3.4 g of cerium nitrate (Ce(NO₃)₃), and 0.15 g of tetraamineplatinum nitrate ((NH₃)₄Pt(NO₃)₂), were added and stirred at room temperature until completely dissolved. Then 2.3 g of aqueous nitric acid (HNO₃) solution (61%) and 23 g of aluminum isopropoxide (C₉H₂₁O₃Al) were added sequentially to the stirred aqueous solution. The contents of Ce and Pt in the above mixture were fixed at 20% by weight and 1% by weight, respectively, based on the final catalyst. The mixed solution was refluxed at 100° C. for 12 hours and then dried at 80° C. for 12 hours. The dried solid particles were recovered and calcined in an oxygen atmosphere at 750° C. for 2 hours. The prepared catalyst was referred as “Pt/CeO₂—Al₂O₃.”

### Example 2: Octane Aromatization Reactivity Measurement

Octane aromatization reaction/catalyst regeneration cycling experiments were performed as follows for the catalysts synthesized in Manufacturing Example 2 and Comparative Examples 2 and 3 above.

2 g of catalyst molded to a size of 200 to 300 μm was placed in a fixed-bed continuous flow reactor and reduced in-situ under a hydrogen atmosphere at 500° C. for 1 hour. The reaction was carried out by injecting a mixture having a hydrogen/octane ratio of 6 under 500° C., 1 atmosphere conditions, and the weight hourly space velocity (WHSV) was fixed at 1 hW. After 6 hours of reaction, the catalyst was regenerated to proceed to the next reaction. Specifically, the reaction/catalyst regeneration cycling experiment was performed as follows:
(1) performing an octane aromatization reaction at 500° C. for 6 hours;
(2) flowing helium at 500° C. for 30 minutes;
(3) regenerating the catalyst with dry air at 500° C. for 60 minutes;
(4) flowing helium at 500° C. for 30 minutes;
(5) in-situ reducing in a hydrogen atmosphere at 500° C. for 60 minutes; and
(5) carrying out the second octane aromatization reaction at 500° C.

The above reaction/catalyst regeneration cycling experiments were performed in total 10 times. The products after the reaction included the main products benzene, toluene, xylene, and ethylbenzene, and by-products obtained by hydrogenolysis and cracking, which were separated and analyzed by gas chromatography, and the yield of aromatic compounds was calculated by Mathematical Formula 2 below.

$$Yield of aromatic compound ( \% ) = \frac{Moles of aromatic compound produced}{Moles of provided octane} \times 100 \qquad [ Mathematical Formula 2 ]$$

The yield of aromatic compound obtained over the prepared catalyst is shown in FIG. 4. As shown in FIG. 4, it can be seen that the Pt/Ce—Al₂O₃ catalyst synthesized in Manufacturing Example 2 exhibited superior aromatic compound yield and catalyst stability compared to the Pt/Al₂O₃ and Pt/CeO₂—Al₂O₃ catalysts synthesized in Comparative Examples 2 and 3.

### Example 3: Analysis of Characteristics of Catalysts after Octane Aromatization Reaction/Catalyst Regeneration Cycling Experiments

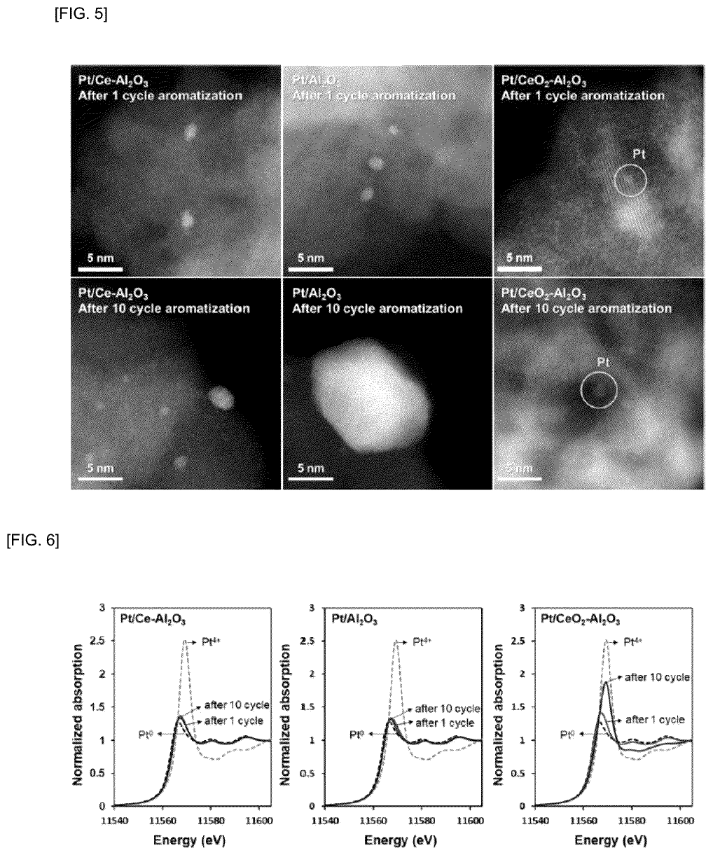

After conducting five octane aromatization reaction/catalyst regeneration cycling experiments on the catalysts synthesized in Manufacturing Example 2 and Comparative Examples 2 and 3 above (Pt/Ce—Al₂O₃, Pt/Al₂O₃, Pt/CeO₂—Al₂O₃), HAADF-STEM analysis and Pt L_{III}-edge X-ray absorption near edge structure (XANES) analysis were performed to confirm the dispersion and oxidation state of Pt, and the results are shown in FIG. 5 and FIG. 6, respectively.

As shown in FIG. 5, compared to Pt/Al₂O₃ synthesized in Comparative Example 2, smaller size Pt particles were found to be present in Pt/Ce—Al₂O₃ synthesized in Manufacturing Example 2. These results can be interpreted that the strong bonding of single-atomic Ce with Pt suppresses the sintering of Pt, resulting in excellent catalytic performance.

As shown in FIG. 6, the Pt/CeO₂—Al₂O₃ synthesized in Comparative Example 3 showed an increase in the oxidation number of Pt as it underwent the reaction/catalyst regeneration cycle, while the Pt/Ce—Al₂O₃ synthesized in Manufacturing Example 2 showed that metallicity of Pt was maintained after repeated cycling experiments.

### Manufacturing Example 3: Synthesis of PtGa/Ce—Al₂O₃ Using Single-Atomic Ce-Doped Alumina Carrier

In 200 mL of distilled water, 0.28 g of cerium nitrate (Ce(NO₃)₃), 0.012 g of tetraamineplatinum nitrate ((NH₃)₄Pt(NO₃)₂), and 0.67 g of gallium nitrate (Ga(NO₃)₃) were added and stirred at room temperature until completely dissolved. Then 2.3 g of aqueous nitric acid (HNO₃) solution (61%) and 23 g of aluminum isopropoxide (C₉H₂₁O₃Al) were added sequentially to the stirred aqueous solution. The contents of Ce, Pt, and Ga in the above mixture were fixed at 2% by weight, 0.1% by weight, and 3% by weight, respectively, based on the final catalyst. The mixed solution was refluxed at 100° C. for 12 hours and then dried at 80° C. for 12 hours. The dried solid particles were recovered and calcined in an oxygen atmosphere at 750° C. for 2 hours. The prepared catalyst was referred as “PtGa/Ce—Al₂O₃.”

### Comparative Example 4: Synthesis of PtGa/Al₂O₃ Using Commercial Alumina as Carrier

0.020 g of tetraamineplatinum nitrate ((NH₃)₄Pt(NO₃)₂), and 1.1 g of gallium nitrate (Ga(NO₃)₃) were dissolved in 6.0 g of distilled water and then dipped in 10 g of γ-Al₂O₃(Strem Chemicals) by wet-impregnation method. The contents of Pt and Ga were fixed at 0.1% by weight and 3.0% by weight based on the final catalyst. After drying at 80° C. for 12 hours, the prepared catalysts were calcined in an oxygen atmosphere at 750° C. for 2 hours. The prepared catalysts were referred as “PtGa/Al₂O₃.”

### Comparative Example 5: Synthesis of PtGa/CeO₂—Al₂O₃ Using Alumina Carrier Containing Ceria

In 200 mL of distilled water, 3.5 g of cerium nitrate (Ce(NO₃)₃), 0.015 g of tetraamineplatinum nitrate ((NH₃)₄Pt(NO₃)₂), and 0.80 g of gallium nitrate (Ga(NO₃)₃) were added and stirred at room temperature until completely dissolved. Then 2.3 g of aqueous nitric acid (HNO₃) solution (61%) and 23 g of aluminum isopropoxide (C₉H₂₁O₃Al) were added sequentially to the stirred aqueous solution. The contents of Ce, Pt, and Ga in the above mixture were fixed at 20% by weight, 0.1% by weight, and 3% by weight, respectively, based on the final catalyst. The mixed solution was refluxed at 100° C. for 12 hours and then dried at 80° C. for 12 hours. The dried solid particles were recovered and calcined in an oxygen atmosphere at 750° C. for 2 hours. The prepared catalyst was referred as “PtGa/CeO₂—Al₂O₃.”

### Example 4: Measurement of Propane Dehydrogenation Reactivity

Propane dehydrogenation reaction/catalyst regeneration cycling experiments were performed as follows for the catalysts synthesized in Manufacturing Example 3 and Comparative Examples 4 and 5 (PtGa/Ce—Al₂O₃, PtGa/Al₂O₃, PtGa/CeO₂—Al₂O₃). 0.65 g of the catalyst, molded to a size of 200 to 300 μm, was placed in a fixed-bed continuous flow reactor and warmed up to 620° C. under a helium atmosphere at a warming rate of 5° C./min. The reaction was carried out by injecting a mixture consisting of 20 kPa of propane and 80 kPa of helium under the condition of 620° C. and 1 atmosphere, and the weight hourly space velocity (WHSV) was fixed at 5.4 h⁻¹. After 1 hour of reaction, the catalyst was regenerated and the next reaction was performed. Specifically, the reaction/catalyst regeneration cycling experiment was performed as follows:
(1) performing a propane dehydrogenation at 620° C. for 1 hour;
(2) flowing helium at 620° C. for 30 minutes;
(3) regenerating the catalyst with dry air at 620° C. for 30 minutes;
(4) flowing helium at 620° C. for 30 minutes; and
(5) performing a second propane dehydrogenation at 620° C.

The above reaction/catalyst regeneration cycling experiments were performed 10 times in total. In addition to the main product, propylene, post-reaction products included by-products obtained by hydrogenolysis and cracking, such as methane, ethane, and ethylene, which were separated and analyzed by gas chromatography, and the yield of propylene was calculated according to Mathematical Formula 3 below.

$$Yield of olefin ( \% ) = \frac{Moles of propylene produced}{Moles of provided propane} \times 100 \qquad [ Mathematical Formula 3 ]$$

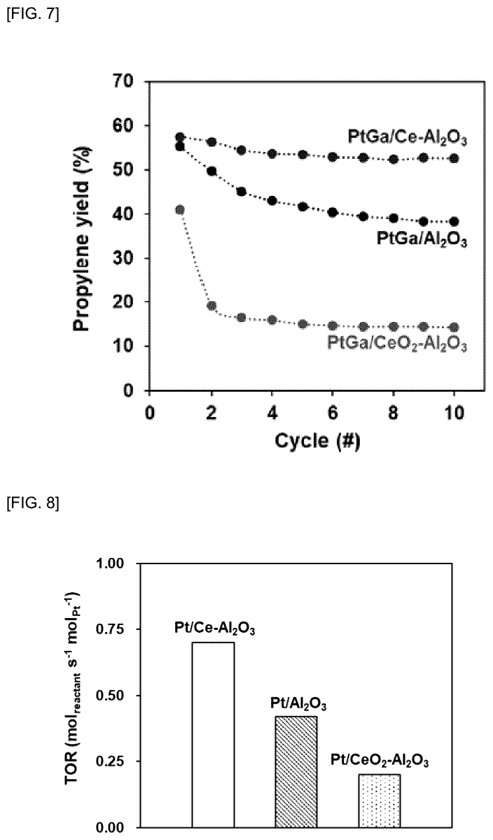

The yield of propylene obtained over the prepared catalyst is shown in FIG. 7. As shown in FIG. 7, it can be seen that the PtGa/Ce—Al₂O₃ catalyst synthesized in Manufacturing Example 3 exhibits superior propylene yield and catalyst stability compared to the PtGa/Al₂O₃ and PtGa/CeO₂—Al₂O₃ catalysts synthesized in Comparative Examples 4 and 5.

### Example 5: Measurement of Benzene Hydrogenation Reactivity

For the catalysts synthesized in Manufacturing Example 2 and Comparative Examples 2 and 3 above (Pt/Ce—Al₂O₃, Pt/Al₂O₃, Pt/CeO₂—Al₂O₃), benzene hydrogenation experiments were performed as follows.

A mixture of 0.1 g of catalyst, molded to a size of 200 to 300 μm, and 1.9 g SiO₂ was placed in a fixed-bed continuous flow reactor and reduced in-situ under a hydrogen atmosphere at 400° C. for 1 hour. The reaction was carried out by injecting a mixture having a hydrogen/benzene ratio of 17.2 at 400° C. and 5 atmospheres, and the weight hourly space velocity (WHSV) was fixed at 526 h⁻¹. Since no by-products other than the main product, cyclohexane, were observed in the post-reaction products, the activity of the catalyst was evaluated as the conversion of benzene per mol of total platinum contained in the catalyst, which was calculated by Mathematical Formula 4 below.

$$Benzene conversion rate ( {mols}^{- 1} {{mol}_{Pt}}^{- 1} ) = \frac{( Flow rate of the reactant \times \frac{Moles of converted benzene}{Moles of provided benzene} )}{( Weight of platinum in the catalyst )} \qquad [ Mathematical Formula 4 ]$$

The benzene conversion rate obtained over the prepared catalyst is shown in FIG. 8. As shown in FIG. 8, it can be seen that the Pt/Ce—Al₂O₃ catalyst synthesized in Manufacturing Example 2 exhibits a higher benzene conversion rate compared to the Pt/Al₂O₃ and Pt/CeO₂—Al₂O₃ catalysts synthesized in Comparative Examples 2 and 3. This can be interpreted that the single atomic-sized Ce present in Pt/Ce—Al₂O₃ interacted with Pt to maintain a higher dispersion while preserving its metallicity.

### Manufacturing Example 4: Manufacturing of Pd/Ce—Al₂O₃

To 200 mL of distilled water, 0.70 g of cerium nitrate (Ce(NO₃)₃) and 0.13 g of palladium nitrate (Pd(NO₃)₂) were added and stirred at room temperature until completely dissolved. Then 2.3 g of aqueous nitric acid (HNO₃) solution (61%) and 23 g of aluminum isopropoxide (C₉H₂₁O₃Al) were added sequentially to the stirring aqueous solution. The contents of cerium (Ce) and palladium (Pd) were fixed at 5% by weight and 1% by weight, respectively, based on the final catalyst. The mixed solution was refluxed at 100° C. for 12 hours and then dried at 80° C. for 12 hours. The dried solid particles were recovered and calcined in an oxygen atmosphere at 750° C. for 2 hours. The prepared catalyst was referred as “Pd/Ce—Al₂O₃.”

### Comparative Example 6: Preparation of Pd/Al₂O₃

0.22 g of palladium nitrate (Pd(NO₃)₂) was dissolved in 6.0 g of distilled water and then supported on 10 g of γ-Al₂O₃(Strem Chemicals) by wet-impregnation method. The content of Pd was fixed at 1% by weight based on the final catalyst. After drying at 80° C. for 12 hours, it was calcined in an oxygen atmosphere at 750° C. for 2 hours. The prepared catalyst was referred as “Pd/Al₂O₃.”

### Example 6: Testing of Sintering Resistance by Calcination Experiment

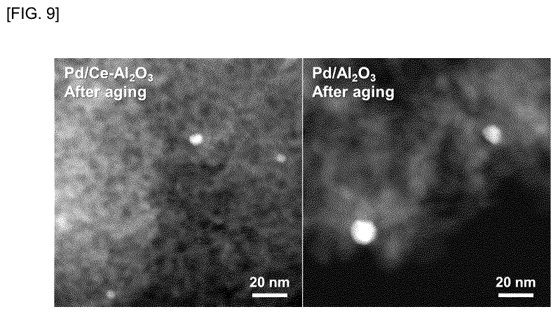

By comparing the degree of sintering resistance of the catalysts synthesized in Manufacturing Example 4 and Comparative Example 6 (Pd/Ce—Al₂O₃, Pd/Al₂O₃), the following experiments were performed to determine whether metals other than platinum (Pt) are also sintering resistant on a single-atomic Ce doped alumina (Al₂O₃) carrier. 1 g of the powdered catalyst was placed in a fixed-bed continuous flow quartz reactor and calcined in an oxygen atmosphere at 800° C. for 12 hours. After the catalysts were recovered after the calcination, HAADF-STEM analysis was performed to confirm the dispersion of Pd, and the results are shown in FIG. 9.

As shown in FIG. 9, it can be seen that the Pd/Ce—Al₂O₃ of Manufacturing Example 4 maintained a good dispersion of Pd even when subjected to the harsh conditions of calcination, compared to the Pd/Al₂O₃ of Comparative Example 6. These results indicate that metals other than platinum (Pt) can be sintered resistant on a single-atomic Ce-doped alumina (Al₂O₃) carrier.

### INDUSTRIAL AVAILABILITY

According to examples of the present invention, by supporting an active metal component on a metal oxide carrier, such as alumina doped with single-atomic Ce, the resistance to sintering can be improved while maintaining metallicity of the active metal.

In addition, the metal catalyst can effectively suppress the sintering of the metal promoted by the high temperature thermochemical reaction, so that it shows good stability in the repeated aromatization or dehydrogenation of hydrocarbons and catalyst regeneration process, and can also show high yield in the hydrogenation.

While the foregoing has described in detail certain aspects of the present invention, it will be apparent to one of ordinary skill in the art that these specific descriptions are merely preferred embodiments and are not intended to limit the scope of the present invention. Accordingly, the substantial scope of the present invention will be defined by the claims and their equivalents.

1. A metal catalyst comprising a metal oxide carrier doped with single-atomic cerium (Ce); and
an active metal component supported on a metal oxide carrier.

2. The metal catalyst of claim 1, wherein the single-atomic cerium satisfies following conditions in Ce_{3d}-XPS spectrum:
(Area of the u′″ peak)/(Total area of the Ce_{3d} spectrum)×100(%)<1(%)
wherein u′″ denotes characteristic peak of Ce⁴⁺ appearing at 917 eV in the Ce_{3d}-XPS spectrum.

3. The metal catalyst of claim 1, wherein content of the single-atomic cerium is 0.01 to 5% by weight based on the metal catalyst.

4. The metal catalyst of claim 1, wherein the metal oxide is at least one selected from the group consisting of alumina (Al₂O₃) and aluminate (MAl₂O₄, wherein M is at least one selected from Mg, Cr, Mn, Fe, Co, Ni and Zn).

5. The metal catalyst of claim 1, wherein the active metal component is at least one selected from the group consisting of platinum (Pt), palladium (Pd), ruthenium (Ru), rhodium (Rh), iridium (Ir), iron (Fe), cobalt (Co), nickel (Ni), molybdenum (Mo), manganese (Mn), gold (Au), silver (Ag), copper (Cu), gallium (Ga) and zinc (Zn).

6. The metal catalyst of claim 1, wherein content of the active metal component is 0.01 to 10% by weight.

7. A method of preparing the metal catalyst of claim 1, comprising:
(a) mixing a Ce precursor, a metal oxide precursor, and an active metal component precursor;
(b) aging a mixture obtained in the step (a); and
(c) drying and heat treating a mixture obtained in the step (b).

8. The method of preparing the metal catalyst of claim 7, wherein the Ce precursor in step (a) is at least one selected from the group consisting of cerium nitrate, cerium fluoride, cerium phosphate, cerium chloride and cerium sulfate.

9. The method of preparing the metal catalyst of claim 7, wherein, in the step (a), the metal oxide precursor in which the metal is aluminum is at least one selected from the group consisting of aluminum alkoxide, aluminum nitrate, aluminum fluoride, aluminum phosphate, aluminum chloride, aluminum sulfate, boehmite and pseudo-boehmite,
wherein the metal oxide precursor in which the metal is a metal other than aluminum, is at least one selected from the group consisting of nitrate, chloride, or sulfate compound of magnesium (Mg), chromium (Cr), manganese (Mn), iron (Fe), cobalt (Co), nickel (Ni), or zinc (Zn).

10. The method of preparing the metal catalyst of claim 7, wherein, in step (c), the drying is performed at a temperature of 50 to 200° C. and the heat treating is performed at a temperature of 350 to 1100° C.

11. An aromatization method using a metal catalyst, comprising performing an aromatization reaction of a saturated hydrocarbon in the presence of the metal catalyst of claim 1 to produce a monocyclic aromatic compound.

12. The aromatization method using a metal catalyst of claim 11, wherein the saturated hydrocarbon is pentane, hexane, heptane or octane.

13. The aromatization method using a metal catalyst of claim 11, wherein the aromatic compound is benzene, toluene, xylene or ethylbenzene.

14. A method of simultaneously performing aromatization and catalytic regeneration using a metal catalyst, comprising:
(a) performing a primary aromatization reaction of a saturated hydrocarbon in the presence of the metal catalyst of claim 1 and flowing helium;
(b) regenerating the metal catalyst with dry air and flowing helium;
(c) in-situ reducing under hydrogen atmosphere; and
(d) performing a secondary aromatization reaction.

15. A dehydrogenation method using a metal catalyst comprising performing dehydrogenation of a saturated hydrocarbon in the presence of the metal catalyst of claim 1 to produce an olefin compound.

16. The dehydrogenation method using a metal catalyst of claim 15, wherein the saturated hydrocarbon is ethane, propane, n-butane, i-butane, butene, or cyclohexane.

17. A method of simultaneously performing dehydrogenation and catalytic regeneration using a metal catalyst, comprising:
(a) performing a primary dehydrogenation of saturated hydrocarbons in the presence of the metal catalyst of claim 1 and flowing helium;
(b) regenerating the metal catalyst with dry air and flowing helium; and
(c) performing a secondary dehydrogenation.

18. A hydrogenation method using a metal catalyst comprising performing a hydrogenation of an aromatic hydrocarbon or an unsaturated hydrocarbon under hydrogen atmosphere in the presence of the metal catalyst of claim 1 to produce an alicyclic or aliphatic compound.

19. The hydrogenation method using a metal catalyst of claim 18, wherein the hydrogenation is hydrogenation, hydrodesulfurization, hydrodenitrogenation, hydrodeoxygenation, or hydroisomerization.
